SEQUENCE LISTING (6 sequences)

SEQ ID NO: 1 - protein, 341 aa, Homo sapiens
  MISC_FEATURE (1)..(341): Accession P16220
MTMESGAENQQSGDAAVTEAENQQMTVQAQPQIATLAQVSMPAAHATSSAPTVTLVQLPNGQTVQVHGVIQAAQPSVIQSPQVQTVQSSCKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 2 - protein, 354 aa, Homo sapiens
  MISC_FEATURE (1)..(354): Accession P63096
MGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 3 - protein, 2505 aa, Homo sapiens
  MISC_FEATURE (1)..(2505): Accession O00555
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAAWGRMPYLDMYQMLRHMSPPLGLGKKCPARVAYKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 4 - DNA, 9752 bp, Homo sapiens
  misc_feature (1)..(9752): Accession NM_004379
tgtttccgtgcgcggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaataaaaaaaatgtctcatttgggaaggaaattccttttaaaaaagagttgagacacttagaaaactaatgttttatatttagtcaagagttatttaagaaagtcaagcttgtttaacaacaaaatatgaagatttaagtgttaattgctggatccattttaaaataagattttaattaacatttgtaaatggtatattttcgtttgtaacaaaccattgtcttttttcaaggatgaacagagtttatgaaggagcatcattctaagaattaagtgatgtagtctttatgtttggacagttcaccagattctcaagaaggctttcaaacaactataaagtttgatgtttgtcctgctgagctaatggggaaagttatagcataaaaattgtgtaaccgcatagatatgtcatttttaaaaactggtttaacagaaatcaagcaaagtcacaaatatgttcacaagttggaattatttattgagtcaaaatgtcgaatcgaacattttgaatgaagtaagtgttataaatgaaaaattgcctgatgtttagcagtttgtattctctaaagctttttttcaaaagttcaggctttctacttactgggaagttggtggtcctcttagtccctgataaatcaaggcaatcacattcatgtgagctggatgaatttataagttataaagaccttatccttcataccttgaggatgattgcactggttttgaagtcagttgcttaatgatgaggtgagaaatgtatcctgttgctaaatctgtcttagacccttggtgaaacttgaagatttcagtttataaagataaaatcaagcatcttttgtgcagttttctttttttaatgcaagaatggtggggaggtttgtttgtaagcatgaaactttgagaatctttattaagaaaatgacataatttttaaaaaccttgtagccaagaacatatgtggccacattaccagtaataaatgtttttctctttatattggccaaaagggaataaaaatgtcatcataggaatttgtacatatgctactgatttgcctagaaaatagcaagtttgatattgctcactttgcaaatatagggccatgtggcacttttatctataggacagattaataaaaatgaagtggggaggggtttatttttgatatattactcttatgagttttcaagctttgataatgtttaactgaaaagtggcttagaaagggctagatccaatgtgttcattattaaataattgctatcagatacaattttaagttcattctttttcaaactcaagtaccatattggcaaccataatattgtcataggtgctctcttcatttagatattcttggggggggtggcatttgtataatatatgtgtacatatatatatatatatatatatatacatacagtatataatctaaagctctgagagctcttaagtcaggaatgctgagtattatagtatattgaggtcagatgaaattttacatttttgtgtgttctgttgcattccttctggtagtttctatgactgcattactccagcactcatgattgattttatcttctaattttcttccaagtattttattttttattagttttctttggcttgatacttttaaatatgttactagtcacttgaaagcctctcccccaaaagtatttggtttgtatgctttgtctgtggcagctataacagtggtaagaacattttgaagatagctttttaaaggaaccactgattttttcaaaaatcatcctgggggaggaattttggcatttcatttgagcagggattttgtcagaaaatgtgttttgatggtaggtcagcagcagtgctagtctctgaaagcacaataccagtcaggcagcctatcccatcagatgtcatctggctgaagtttatctctgtctctcaggataaatccctgtaggacaaatccctactatcatttctaccttttggggtgacatgtggaatcatacaaaggcttaggaagaaatacgtttgtttaaaccaggatgctttacttacttgaagtgacttcaatctagatttcttttaatatttaacaaatttttaattctatgatcagccacagtcagctattaccataaattggtctctgtttattttgaagatcacggctgcttcattttgcaggattaagtagggctaatgtatcttaaagttaagatcttgaattaaagtgagttttagaaatagtgttacataccttttcagttgttttcaagaggctttatttttgttgcctttgtagccctgaaagctgttggtatattttttccctcatggacccaatagaaaagttgtatatttatttggattatatttacattctgtcctttgtaaatgtttggtgtaacttgcacttttttaaatgacccagtttgggtattagcaacttaagaaattccctcatcaagtaattctcaactttttagtctttctcctctcttcaaatcatgtgactttttaaatggaagtttttcattgattaaaatattttagcacctaaaagctagccttaaaaacagctgtaaaagaaaaacatcaggaaattagatatgactagcccagttaattaaaagacgggctcaaaccttgttttattctttttcatcttggatgaagattgaagggaaaataactcaagtgcataatatttattttcaatttttaatgagactttatcctcatcacaacattaatactgtacatagtatgccaaaatatccattaatttgtctagaatagtacaagactttttaaagcaattgtcctcacagagaccacatgtaatatactgaaatatgttcatttttaatggctttgttaacatcaaagaaatgctgcctaaatttgatttcagatgaggaaggagaaagtaaagtgtgcatagtaaggctgtaggtgaagagttgtgagataaatagttcactcagttgtacaaagcacaactagaactttttgttgggaggcttacatacatcttgaatattcttaatgtaataatgttgactattaagttggctacacagtcactgtatgtactaggaactggtttccttgacattctagaatcaatggctaggagaggcattaatctttgaggggctgaacatatcatgaagctgagtcagtatggaaaattttcaaataaacagggtgctgaagttccatctgtctcatctgcttatgataagttcttattgattagtgaatgtagcttaagcctttgtatgtgtcctcagggggcagaccgactttaagagggaccagataacgtttgaatggagggattatatttcaggtgttttagcttgaaatttattttttaaaaaaagaaaaatttaaaaaatatataaataaaatagaacaaagccggtgatgcaagttgatattataaacaggcagttttagcacagaaagaaaatactgacctgtctgcattctggtacggtgggtgcaggtcccagctgggtatgacatgatacatttttaattattctcaccagcaagtaaaaggaaaatgaacaatcttttggaattgtctttgaaaaggatcaaagagtaggaaattcacatttgacctaacattacttgcctatagaagtatggcatttccaagcttttgtctgaggagcatctcagagaagtgagagtaaatctgagttagcttaaaaattggtagggaggaagaaaatctctgcaaataatgattttatgtttgttggccaagtgaaatgatctatcattgtgtttgggaggttttattttcttatgtttttaaaattggtaaatgctttatagatgtatttttatccaagtgccactccaatttgtgtatgtaataaaattatttatattaaaagtgggaaataattgtcaacattttttttgagtatagatttattaggggtggcaaagaagagtgctagttagcagttttccatgtaaagttgtccttgactgatttgtccacatgtcagttgtaactcccccactccctgcaaaaggaattatttctaacccagatgtatcacttgaaactttttagaagcaaaataatcagggaagttcctagaaaggtgtttggctttttggtttttgagggttggggtaaagaagacttcccccacaactgtcagcacaaaacagggtattgatttttaactctgatgtttctattggagttgaatactaaataaataactataatgagggaaatacatttctaataaaattccctacattctagaaacatccctgttttaatttttttatctaaatctttttgtgctttatgtgtaaagaaaaaaatgtactgagttacaatgcattttattaacactatgtacataatagctgctttgtgttcagaatagtagcagttgctttgtatattaaagtgatccttgtgaatttgtgaaatattgtcataaagtgctttttcttactgtaatctttgtggtatcaactgtcataatgctctttttacacaaacatttatgtgcagtcacataaacatgcttttaaaaactctgtaagtctcttttttggggatgggatctctatattttgttgggttttttttgctagtagtgtgaagccatgttttattggacttaaagttacaatatattacaagcttgtgttggaaggcagcaaaactaattcagacaacaacatgtcttcagttactggatccctaattttcaggacaaaacctgtttttcaataagattgaacagtgcctatttgtggatttggagatgttactgtcaagatgactaatggagacatacgaccagctgtgtctgatgtcataaaacacgtgttcactgaaaggacaataagactatataccttctcaggtccccttgcaattctaaaactctgtgatcatataaattggaaggaaaggggaggggatatggttaatctttgcttaagctgtaagaataaaaaagttatctcctatactattaacttctgaaataagttctgagacgagacatctgaaaataagcagctgcattatttgtatgtttcttcactgccaagatgtgttcaagcctgctatacctgccattgtattggaaggcttaatgaatttcatttattttctgcaacaacgattacagaatttattgcacaaaatgagacattttgagagtgatattaattacatgagggacaataggcatgaactaggattgttctaagcaaatcggaatcgggtcaccctgccacgttcaggtgcttggaccttcaggaaaagattgcccatcttgtcatttgaccaggcactgaagtgacaagaccatccttgagaagtcacatccaaagataaaattctgatccatttctagttttagtgtttcgccactgaagacttaacatatgtcttttacactcaggttgcaaaacacaggcccaagacaaacttaacttctcccccaaatcttccttccgctggtttttccatctcgtaagtggtgccactatccatctgttaaattgtttaggggaaacctagaaaagcactaccttaatcagtgttatccttcttcttaactgtgcgtcctaatttctccacatctttcttaagtgcagtgaccaaaccggatgagaattctaacacgggcctgacatcaaatggaaaggaaggataatgtccaggagttggaatgttatccttgtttttaattaagatgcaattcacataaattaactttttaagtgaacaattaagtggtagtacatccacaatggtgtacaaccaccacttctatctagctccaaaacattctcatcactccaaaagtaaagtcccgttactctccattttctcctcccaccgcccttgtccctggcaaccaccaatctgcttcctgtttctttggatttacatccgggtatttcatgtgagactcatacactgtgtattacttctttcgtctagctttaatgtgttgttgaggttgatccattgtaacatgttatcactacttcattcctttttatagctaagtatactttttatagtaagtatgccattgtagatatataccacaagtttatcgattcatccagttgagttgtttctactgtttggctaatgttcatagtgctgttatgaatgttcgtgtacaagtatttgagtccgtgttttcaattatttggggtatatgcctgggagtggagttgctgggtcatgttgaaatcgcacatttaactttttgaggaactgtcaaactttccctcagcagctgtaccgttttaccttccaccattgatgtatgagggttccaatttctccacaccttcaccaacacttattttgccattttaaaaattatagccatcctcatgggtgtggtctctcattgtggttttgatttgcatttccctgattactaatgatgtggagcatcttttgttgtctttggccatctgcgtatcttctttgaagaaatgtctgttgaggtcctttgttcattgaaattttgttgttgggttctgagttccttatatattctgggtactaggcccttataatattttcgcctataagtttttgctttataatgtcctcattgttttcaaacttactttatgtaatatgtacacttctaaaaaaaagaaacatggaaaagggcaaactgtaaaaaaaaaaaaaaaaa

SEQ ID NO: 5 - DNA, 3318 bp, Homo sapiens
  misc_feature (1)..(3318): Accession NM_002069
ggaggccgagctcggctgggcttggcgaggctgcggcgcggccaccggcgggagtgcagcggccactgtacccagagattcaaaaccccaaacccgggacttgggggcgctgagccgggccgggaagcagagcctggtcgtgaggaacagccgcccgttgctgtctgcccctttgcggacagcgtctccctcgactccgcttaggaagtggtgggggcggcgtggcccccgtcgggaggcgttcgaacgcccgctaggagagagaaaggattcccctgtgcttggagcccgcactcgggcgcggagggagcggcggcaggctctcgctttcggcaccatgggctgcacgctgagcgccgaggacaaggcggcggtggagcggagtaagatgatcgaccgcaacctccgtgaggacggcgagaaggcggcgcgcgaggtcaagctgctgctgctcggtgctggtgaatctggtaaaagtacaattgtgaagcagatgaaaattatccatgaagctggttattcagaagaggagtgtaaacaatacaaagcagtggtctacagtaacaccatccagtcaattattgctatcattagggctatggggaggttgaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtgacggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctctttgaagaaaaaatcaaaaagagccctctcactatatgctatccagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaagttttgcagttcatggtaaaatgcattttcaaaccaaatgagtacttatatatggatctctgtagactagagtcttgcagcaacacagaatgtaatataaggcaaatgcatctgggacttgaccaaagttgttctgttttgtttttttaactgaaagtaacagaaggacctttcttaaatgtgacagatggtcctgcagtgtgaaactgaaggacagtgttaaagctgggctctagtatattgatgatttctgcataagtgtaaatatgcaaatgtatgtatacatgtatttatgactttagttttcgacattatttttaggttttaagagtggcaacttaggattttagggtgatggctttggaaataacataaatataccttgtactgaatgacagactattactacgtttgccagttttaaacagctttatttatgttcatgtcctgtaaatttttaagtacagtaattaatattaggaaacattacagcccttatctagattatatgtatacttgtattaataaaaatgttatttgtacaaacattgcacagactattttaataacatgatttgttctttaaattttatgtgttttattgaaatgttcttaagatgaatacacctgcctttggatcaactatttaaacattgtatgcattttgatttttcctactttaagaaaataaaataatttaattttacattagattccacgttagatttggtttgaaaaactaaaatttcagatttctgaggatatactgtcttagacttattgtacacacttagtttttattcacttgttttcactctgaattttaatatttggctgatatgaatgcattgcctcaaaggtgatgtcatcttaatttttattcactttaaataactacatttttgtttataactaagtttggagggatcctaagagcatttttgtgggtaaaaaaaaaacctgtggacataatgaattttgagacattgattggtgaggcttttatttcccttgaggagtctcttgtacctagcatacatgatagctccttgttgggaagataacaagaaggatctttgaatactctattgctgatataatgcaagatttaaatttatacatataactaatttcaaatgtaattatcacactatgttaaaattacttttttcccttagataattcaaatttctccacttgcttgagattatatcatttctttttcaattatactattatttctgagaatgaaatggacgattacacttagaaaatgagtaatagtgtttaataagtcagtgattatatgtgtgctcaaataagtgttatgtatcagctagatactgagctttgatagaataattttcttttgattattcatgatgtgtcatctctgaccttgtttcagcaaagtaaacagcactccccaccccaccctcctttttttactcatcttggaaaaggttagtctttcagtacacgttgctggtaagtagtttccaagttacgtgttgtcactgggttgaagtatatttgtgtgtgtgtgtgtgtgtgtgtgtgtgtgtgtaaccataaactatattcatatctgtttcatttggaggattttcttctttgtaatgtaaagaaattcaaagttatcaaagttccttaaatgtgttagtttagattctttatgtgcctttcatgaaagatatgttttcattaattttactggtggacctgtaatatccacattgtgaagctgtgtatgaaattcaactataatatgaataaatttgaatcatgagaattatgggttaaaaagccacaaagaagcacatattggtgaccatcattaatgaaatcctgaactttattctgtgtaattgtgttaataaatcctaataaatttaaatttttaaaattttacaaac

SEQ ID NO: 6 - DNA, 7446 bp, Homo sapiens
  misc_feature (1)..(7446): Accession X99897
cgcaccctccttccgcccctccttctccggggtcagccaggaagatgtcccgagctgctatccccggctcggcccgggcagccgccttctgagcccccgacccgagcgccgagccgccgcgcgatgggctgggccgtggagcgtctccgcagtcgtagctccagccgccgcgctcccagccccggcagcctcagcatcagcggcggcggcggcggcggcggcgtcttccgcatcgttcgccgcagcgtaacccggagccctttgctctttgcagaatggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggaatggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggaggacccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggtcccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacggcagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagtgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgtggcttatgaaaatgccctgcgggtgttcaacatcgccttcacctccctcttctctctggaatgtgtgctgaaagccatggcttttgggattctgaattatttccgcgatgcctggaacatcttcgactttgtgactgttctgggcagcatcaccgatatcctcgtgactgagtttgggaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttgcggtcggattcattataaggatatgtacagtttattacgagtaatatctccccctctcggcttaggcaagaaatgtcctcatagggttgcttgcaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcaggcggtggccaggccggccgggcggccaccagcggccctcggaggtacccaggccccacggccgagcctctggccggagatcggcgcccacggggggccacagcagcggccgcacgcccaggatggagaggcgggtccaggcccggcccggagcgagtctccagggcctggtcgacacggcggggcccggctggcggcagtc

(19) United States
(12) Patent Application Publication
(10) Pub. No.: US 2010/0099741 A1
(43) Pub. Date: Apr. 22, 2010

(54) Molecular Targets for Treatment of Learning and Memory Dysfunction

(76) Inventor: Effat Emamian, New Milford, NJ (US)

Correspondence Address: MCCARTER & ENGLISH, LLP HARTFORD;CITYPLACE I, 185 ASYLUM STREET, HARTFORD, CT, 06103, US

(21) Appl. No.: 12/582,547

(22) Filed: Oct. 20, 2009

Related U.S. Application Data
(60) Provisional application No. 61/106,886, filed on Oct. 20, 2008.

(51) Int. Cl. A61K 31/7052; C12Q 1/48; C12Q 1/02; C12N 15/63; A61P 25/00; A61K 31/554; A61K 31/495; A61K 31/4439; A61K 31/44; A61K 31/40; A61K 31/277

(52) U.S. Cl. 514/44 A; 435/15; 435/29; 435/320.1; 514/44 R; 514/211.05; 514/255.04; 514/338; 514/356; 514/428; 514/523

(57) ABSTRACT

Described herein are methods for identification of alternative safe molecular targets and novel regulators in complex molecular networks using a novel fault diagnosis engineering approach. For example, in this invention we claim new molecular targets that could be effectively targeted for changing the activity of CREB, and therefore for treatment of learning and memory related disorders. Learning and memory dysfunction is a major clinical manifestation of a number of human disorders, such as Alzheimer Disease (AD), schizophrenia, dementias, autism, etc. More specifically, we claim that composition and compounds that can target the activity of L AND/OR P/Q-, N-type calcium channels, Gαi, Gβγ, PP2A and CaMKII and IV are effective therapeutics for the treatment of disorders manifested by learning and memory dysfunction.

### CROSS-REFERENCE TO RELATED APPLICATIONS

This application clams the benefit under 35 U.S.C. §119(e) of U.S. Provisional Patent Application Ser. No. 61/106,886; filed: Oct. 20, 2008, which, along with PCT/U.S.08/054,674, are hereby incorporated by reference their entirety.

### INCORPORATION BY REFERENCE

In compliance with 37 C.F.R. §1.52(e)(5), an electronic CRF of the sequence listing is filed herewith: file name: Emamian_{—}4_seqlist_ST25.txt; size 58 KB; created on: Oct. 20, 2009; using PatentIn-3.5, and Checker 4.4.0 is hereby incorporated by reference in its entirety.

### FIELD OF THE INVENTION

The present invention relates to molecular targets and methods of modulating their activity for the treatment of diseases and disorders, in particular, learning and memory related disorders.

### BACKGROUND

We recently developed a technique that enables the identification of novel regulators in complex cellular networks (1). This techniques is capable of measuring the functionality levels of numerous molecules in complex molecular networks and compare the functionality levels of several molecules in the cell. This technique provides a means of identifying alternative safe molecular targets for those molecules that cannot be directly targeted due to the severe risk of development of toxicity.

Presently, we describe the identification of several novel critical regulators of cAMP responsive element binding protein (CREB), and their use as targets for the identification of therapeutics for the treatment/prevention of learning and memory-related disorders.

### SUMMARY

In neurons, appropriate long-term adaptive responses to changes in the environment require the conversion of extracellular stimuli into discrete intracellular signals. Many of these signals involve the regulation of gene expression. The cAMP responsive element binding protein (CREB) is a nuclear transcription factor that modulates transcription of genes containing cAMP responsive elements (CRE sites) in their promoters (3). CREB is a key part of many intracellular signaling events that critically regulate many neural functions. Numerous studies on invertebrates and vertebrates demonstrate that CREB is critical for long-term memory (3). CREB-dependent transcription has a critical role in different forms of plasticity, including long-term memory in mammals. Several human cognitive disorders have been linked to alterations of CREB-regulated gene expression (3).

The present invention relates to methods for targeting and modulating CREB function in the development of novel treatment strategies for human disorders manifested by learning and memory dysfunction. Traditionally, however, targeting CREB directly, by small molecules or chemical entities, has high risk of toxicity due to the critical physiological functions of CREB. Using the methods of the present invention, previously unknown regulators of CREB activity can be targeted instead, which can lead to the identification of therapeutics, which have less chance of producing drug toxicity.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention.

FIG. 1. Western blot analysis on the protein extracts from primary neuronal culture after 2.5 and 12 hours treatment with either vehicle or approximately 1 μM concentration of the selective P/Q-type calcium channel blocker, ω-Agatoxin IVA. Each blot shown in this figure is a representative blot of three independent experiments.

FIG. 2. 48 hours following adenoviral transduction using three unique shRNA constructs targeting P/Q-type calcium channels. Lane 1 shows the protein size by a protein marker (Magic Marker from Invitrogen). Lanes 2 and 6 are loaded with protein extracts from two control plates (mock) and lanes 3, 4 and 5 are treated with V1, V2, and V3 adenoviral vectors, respectively. Top blot shows a decrease in P/Q-type calcium channel abundance with V2 vector (lane 4) and a smaller decrease with the V1 vector (lane 3). Middle blots show the total protein and Ser¹³³ phosphorylation of CREB in the same set of samples. The same membrane of the top blot was stripped and re-probed with anti-CREB antibody. Bottom blot shows total actin protein of the same membrane, as the loading control.

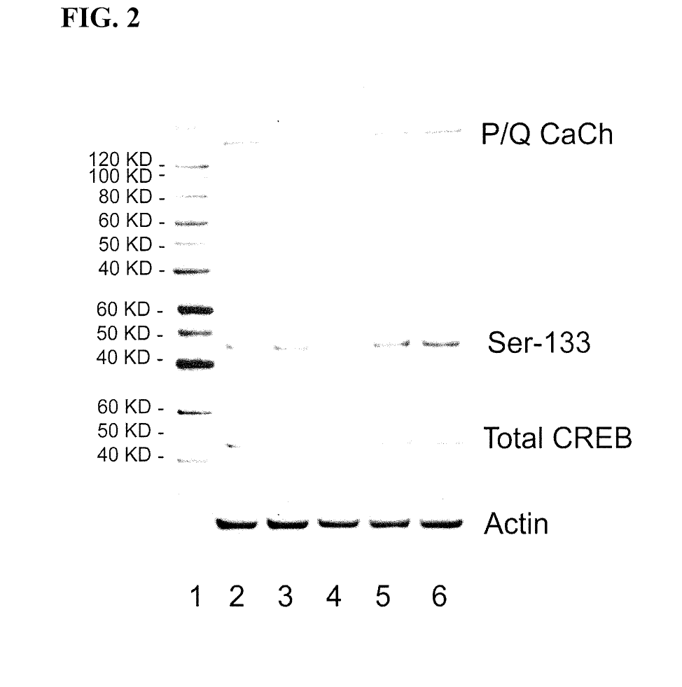

FIG. 3. After 2 hours treatment with approximately 0.1 or 0.2 μg/ml of the Gαᵢ antagonist, pertussis toxin, and approximately 5 or 10 μM of Gαᵢ agonist, MAS-7. Blots were first probed with antibody against phospho-CREB (Ser¹³³) (top) then stripped and re-probed with antibody against CREB (middle) and the antibody against actin (bottom), as the loading control for confirming equal loading.

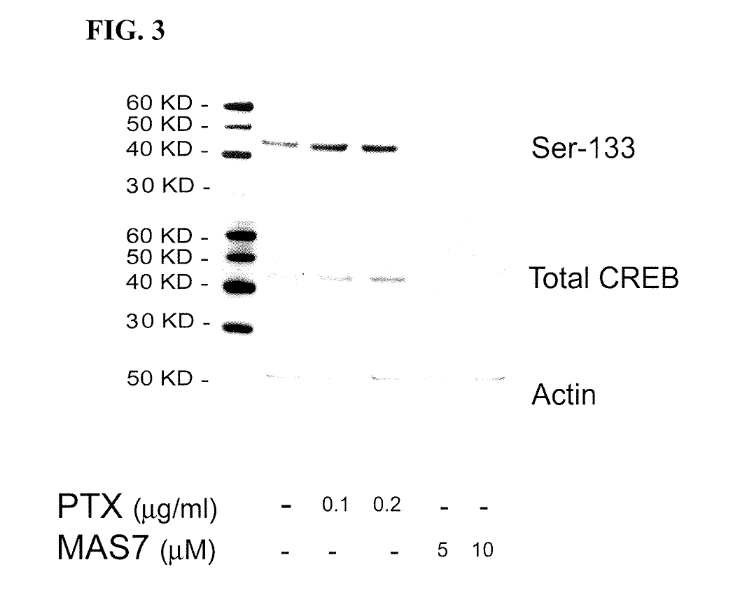

FIG. 4. The activity of CREB following treatment with different neurotransmitter ligands, including serotonin (STN), glutamate (GLT), dopamine (DPM), GABA, and adenosine (ADN). Top blot shows phosphorylation of CREB at Ser¹³³, 15 minutes after treatment with vehicle, 10 μM forskolin (FSK), serotonin, glutamate, dopamine, GABA, and adenosine, respectively. Middle blot shows total CREB protein on the same blot. Bottom blot shows actin as loading control.

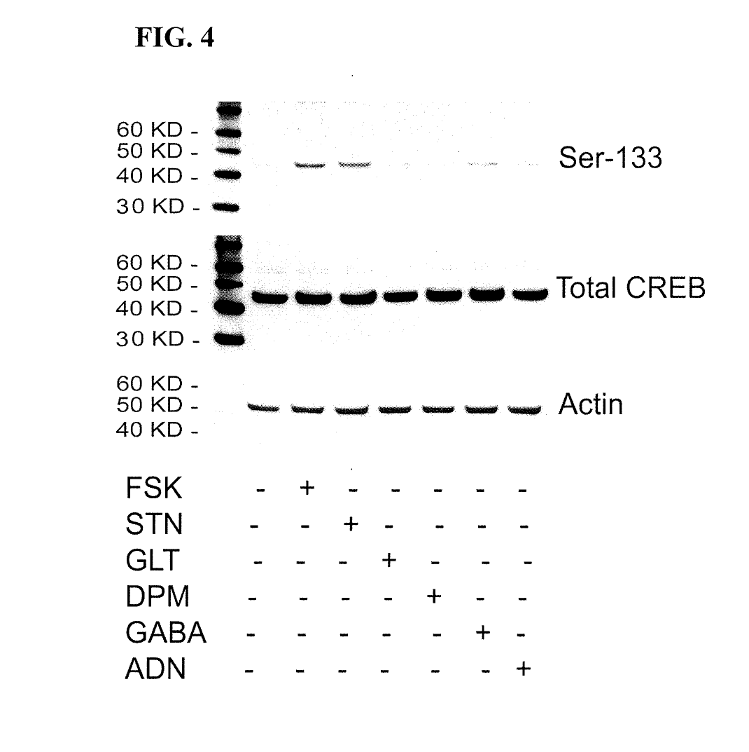

FIG. 5. The effect of treatment with serotonin on the CREB activity when PKA is activated with forskolin or inhibited with H-89. Cells were treated with vehicle, about 10 μM forskolin or H-89 for 30 minutes followed by 15 minutes treatment with about 10 μM serotonin.

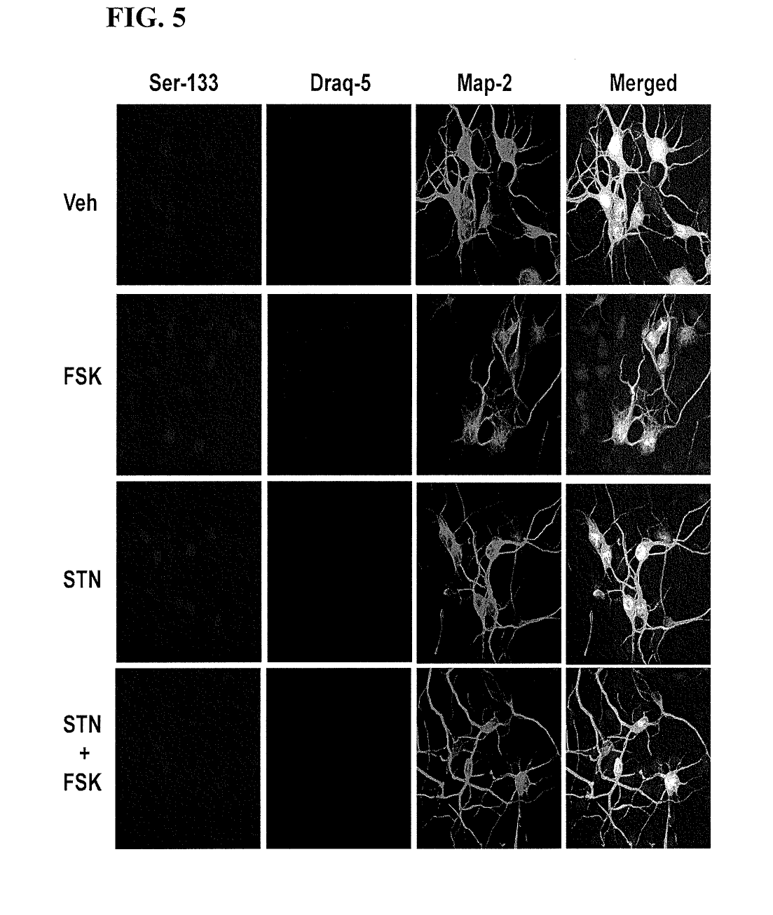

FIG. 6. Immunofluorescent analysis of primary cortical culture following treatment with vehicle, forskolin, serotonin, or serotonin and forskolin. Red represents the phosphorylated CREB (Ser¹³³) as a measure of CREB activity. Green represents the Map-2 staining as a specific neuronal marker and blue represent DRAQ-5 nuclear staining of all the cells in the primary culture (both neuronal and glial cells). Images were captured with the same confocal parameters for the four different treatment conditions.

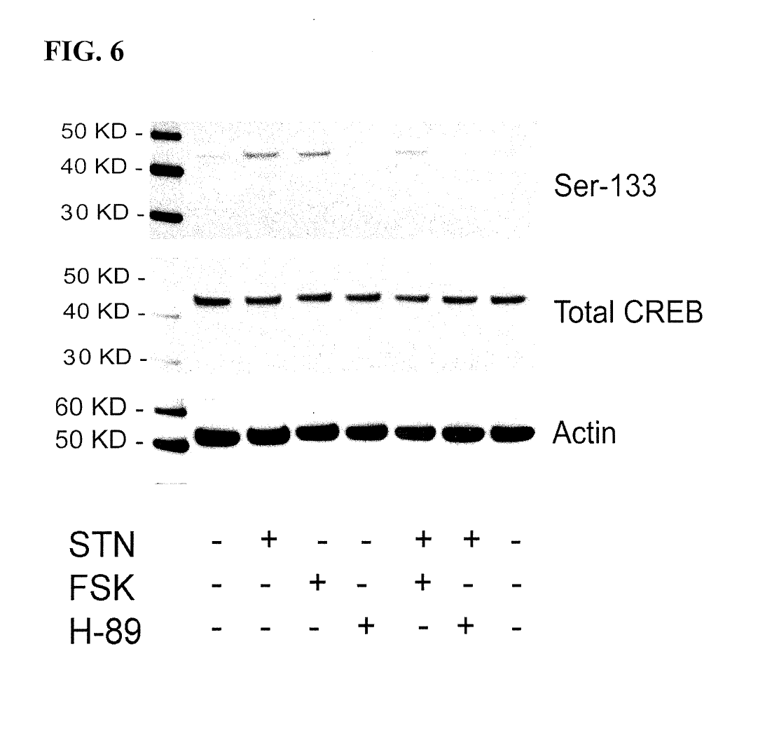

### DETAILED DESCRIPTION

The present disclosure provides advantageous systems and methods for identifying molecular vulnerabilities in biological pathways. U.S. Provisional Patent Application Ser. No. 61/106,886; filed: Oct. 20, 2008, and PCT/U.S.08/054,674, are hereby incorporated by reference their entirety.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In the case of conflict, the present specification, including definitions, will control. In addition, the materials, methods, and examples are illustrative only and not intended to be limiting.

The present invention describes compositions and methods which are surprisingly and unexpectedly beneficial for diagnosing, treating, and/or preventing neurological disorders or trauma, and/or learning and memory dysfunction.

The present disclosure generally involves conceptualizing a disease/disorder at the molecular level as a faulty physiological system, wherein one or more molecules in the complex intracellular network are dysfunctional. Recent progress in genome- and proteome-wide expression analysis of biological systems provides a valuable picture of “expression levels” of molecules. In the disclosed systems and methods, it is the “functionality” of each molecule which determines the overall performance of the molecular system. Thus, molecular fault models—similar to some fault models used in digital circuits—are developed according to the present disclosure in order to quantify the functionality and dysfunctionality of different molecules in a network.

Thus, the disclosed conceptualization is accomplished by modeling a given physiological system as a digital logic circuit. More particularly, in exemplary embodiments, binary logic equations are derived by analyzing the interactions between the input and output nodes of a biological system of interest. These equations are then used to produce a digital circuit representation for the system. Once a digital circuit representation is created, this model may advantageously be analyzed, in order to determine molecular vulnerabilities.

In order to determine the molecules of greatest vulnerability, fault diagnosis techniques, e.g., vulnerability assessment methods disclosed herein, are used. Such vulnerability assessment methods provide numerical representations of the functional vulnerability for the entire molecular system as it relates to each individual molecule. Thus, a high vulnerability value for a molecule indicates a high probability that dysfunction of said molecule will disrupt the system and thereby change the system from a physiological state to a pathological state. Identification and isolation of such molecules, e.g., having high vulnerability levels, is a major step towards understating the molecular source(s) of a variety of diseases. From a drug development perspective, vulnerability assessment provides and/or helps to define/identify a set of candidate molecules to target for regulation.

Taken together, our experimental data indicates that fault diagnosis engineering can identify new critical regulators, and also correctly predict previously known regulators of the output molecules. Furthermore, we have provided experimental evidence that a reconstructed Boolean network can correctly predict the activity of output molecules, based of the activity levels of input signals. Finally, our experimental data supports the theoretical finding of the proposed fault diagnosis approach, specifically the experiments confirm that for proper propagation of the input signals to regulate the activity of output molecules, the normal function and activity level of highly vulnerable molecules are necessary. When these highly vulnerable molecules become faulty, stuck-at-1 or 0, the interconnected pathways can not correctly propagate the signals from input to the output, and the molecular network does not function properly.

Using the system and methods of the invention, the inventors have identified molecular entities in the regulation of CREB (cAMP response element-binding) that were previously unknown. See, accession number: P16220 (human CREB 1; SEQ ID NO:1), which is incorporated herein by reference.

More specifically, the inventors have identified G_{αi} and calcium channels as novel regulators of CREB. Notably, the system and method of the invention also identified a number of molecular vulnerabilities that have been previously shown to be the main regulators of CREB, including, cAMP, AC1, AC2, AC5 and PKA. The identification of entities that have been shown, through empirical evidence, to regulate CREB shows the predictive accuracy of the system.

CREB proteins are transcription factors which bind to certain DNA sequences called cAMP response elements (CREs) and thereby increase or decrease the transcription of certain genes. CREB is highly related (in structure and function) to CREM (cAMP response element modulator) and ATF-1 (activating transcription factor-1) proteins. Activation of CREB occurs in response to external stimuli, for example, the activation of a membrane surface receptor. The activated receptor, in turn, leads to the activation of effector molecules leading to the production of cyclic-AMP or Ca2+ release, which leads to the activation of a protein kinase. This protein kinase translocates to the cell nucleus, where it activates the CREB protein. The activated CREB protein then binds to a CRE region, and is then bound by a CREB binding protein (CBP) which coactivates it, allowing it to switch certain genes on or off. The DNA binding of CREB is mediated via its basic leucine zipper domain.

CREB proteins play a major role in regulating gene expression in neurons, and are believed to be involved in long-term potentiation (i.e., long-term memories). CREB is also important for the survival of neurons, as shown in genetically engineered mice, where CREB and CREM were deleted in the brain. If CREB is lost in the whole developing mouse embryo, the mice die immediately after birth, again highlighting the critical role of CREB in promoting survival. Moreover, disturbance of CREB function in brain can contribute to the development and progression of Huntington's Disease, Alzheimer's, Rubinstein-Taybi syndrome, cancer, as well as other neuropathologies such as schizophrenia, anxiety disorders as well as the other psychiatric disorders. See also, Barco A, Bailey C, Kandel E (2006), “Common molecular mechanisms in explicit and implicit memory,” J. Neurochem. 97 (6): 1520-33; Conkright M, Montminy M (2005), “CREB: the unindicted cancer co-conspirator,” Trends Cell Biol. 15 (9): 457-9; Mantamadiotis T., et al., (2002), “Disruption of CREB function in brain leads to neurodegeneration,” Nat. Genet. 31 (1): 47-54; Mayr B, Montminy M (2001), “Transcriptional regulation by the phosphorylation-dependent factor CREB,” Nat. Rev. Mol. Cell. Biol. 2 (8): 599-609; Yin J., et al., “CREB as a memory modulator: induced expression of a dCREB2 activator isoform enhances long-term memory in Drosophila,” Cell 81 (1): 107-15, which are incorporated herein by reference in their entirety.

As indicated previously, one of the molecules that has been identified using the system and methods of the invention as being a vulnerable molecule in the dysregulation of CREB is the guanine-nucleotide binding protein (i.e., G-protein), G_{αi}. See, accession number: P6309 (human Gai; SEQ ID NO:2). G proteins, are a family of heterotrimeric proteins involved in second messenger cascades. The dysfunction of G_{αi} has not previously been implicated in the aberrant modulation of CREB activity.

Heterotrimeric G-proteins are activated by G protein-coupled receptors (GPCRs) and made up of alpha (α), beta (β), and gamma (γ) subunits. G-proteins function as molecular switches, alternating between an inactive, guanosine diphosphate (GDP) bound state, and an active, guanosine triphosphate (GTP) bound state. Receptor-activated G-proteins are bound to the inside surface of the cell membrane. They consist of the G_{α} and the tightly-associated G_{βγ} subunits. At the present time, four main families exist for G_{α} subunits: G_{αs}, G_{αi}, G_{αq/11}, and G_{α12/13}. These groups differ primarily in effector recognition, but share a similar mechanism of activation. When a ligand activates the GPCR, it induces a conformation change in the receptor that allows the receptor to function as a guanine nucleotide exchange factor (GEF) that exchanges GTP for GDP on the G_{α} subunit. In the traditional view of heterotrimeric protein activation, this exchange triggers the dissociation of the G_{α} subunit from the G_{βγ} dimer and the receptor. However, certain studies also suggest that G-proteins function through molecular rearrangement, reorganization, and pre-complexing of effector molecules. Furthermore, both G_{α}-GTP and G_{βγ} can activate different signaling cascades (or second messenger pathways) and effector proteins, while the receptor is able to activate the next G protein. Once in its active state, the G_{α} subunit will eventually hydrolyze the attached GTP to GDP by its inherent enzymatic activity, allowing it to re-associate with G_{βγ} and starting a new cycle.

The class of G-proteins known as G_{αi} are known to inhibit the production of cAMP through an antagonistic interaction with the enzyme, adenylate cyclase. G_{αi} is known to complex with several GPCRs including the 5-HT (serotonin) Receptor type 1, Adenosine Receptor A1 and A3, and Prostaglandin Receptors. Interestingly, the G_{βγ} subunit of G-proteins are also known to couple to L-type calcium channels, affecting their voltage activation/inactivation, single-channel conductance, and/or open probability.

Fault diagnosis engineering analysis of the CREB pathway identified G_{αi} as a highly vulnerable molecule in the CREB pathway. To date, there have been no reports showing that G_{αi} can modulate the activity of CREB. As such, the inventors contemplate methods of modulation of G_{αi} activity, either directly or indirectly, for the treatment or prevention of neuropathological disorders, for example, memory loss, Parkinson's, Alzheimer's, Huntington's disease, schizophrenia, dementia and other neurodegenerative and psychiatric brain disorders manifested by learning and memory dysfunction. In addition, the inventors contemplate methods for the activation of pathways which antagonize G_{αi} activity, for example, the activation of G_{αs}, or the activation of adenylate cyclase as a means of treating CREB-related disorders. Moreover, the inventors also contemplate methods in which the activity of G_{αi} is measured as a means for identifying and/or screening potential therapeutic compounds. Furthermore, the inventors also contemplate the use of G_{αi} activity, gene or gene expression as a means of detecting or diagnosing a disease state, for example, the presence, absence or severity; or determining an individual's predisposition to developing a neuropathological disease related to CREB dysfunction.

Fault diagnosis engineering analysis of the CREB pathway performed according to the methods of the invention have also identified for the first time that the P/Q type calcium channel is a highly vulnerable molecule in the CREB pathway. See, accession number: O00555 (human P/Q type voltage-gated calcium channel; SEQ ID NO:3). This means that the functionality of CREB is closely correlated to the activity level of P/Q type calcium channel. Therefore, the system and methods of the invention have also implicated, for the first time, the dysfunction of the P/Q Calcium Channel in CREB-related neuropathologies. The P/Q channel is an L-type, voltage-gated calcium channel, and is the main channel involved in nerve evoked neurotransmitter release at neuromuscular junctions (NMJs) and many central nervous system synapses. The P/Q channel becomes activated (i.e., opens) in response to membrane depolarization, for example, from the binding of a neurotransmitter to its receptor. Once activated, the P/Q channel allows calcium ions to enter the synapse. Calcium also acts as a second messenger, triggering neurotransmitter release/uptake as well as inducing changes in gene expression.

As indicated above, it has been shown that G-protein beta-gamma subunits can interact with L-type calcium channels. Therefore, is remains possible that G_{αi} dysfunction is compounded by inducing aberrant regulation of P/Q channels, which may further lead to dysfunction of CREB. As such, the inventors contemplate additional methods of modulation of P/Q channel activity, either directly or indirectly, for the treatment or prevention of neuropathological disorders, for example, memory loss, Parkinson's, Alzheimer's, Huntington's disease, schizophrenia, dementia, and psychiatric or neurological conditions manifested by learning and memory dysfunction. Moreover, the inventors also contemplate methods in which the activity of the P/Q channel (i.e., open probability, activation/inactivation kinetics, channel current, conductance, and the like) is measured as a means for identifying and/or screening potential therapeutic compounds. Furthermore, the inventors also contemplate the use of the P/Q channel activity, gene or gene expression as a means of detecting or diagnosing a disease state, for example, the presence, absence or severity; or determining an individual's predisposition to developing a neuropathological disease related to CREB dysfunction.

Furthermore, the inventors contemplate methods in which agonists or antagonists of the P/Q, and/or L type channel and G_{αi} are administered simultaneously for the treatment and/or prevention of neuropathological disease related to CREB dysfunction. Many calcium channel and G_{αi} agonists and antagonists are currently available (e.g., omega-conotoxind, omega-agatoxin, verapamil, diltiazem, DHP, roscovitine, nifedipine, atropine, Bay K 8644; NEM, forskolin, cholera toxin, pertussis toxin, respectively), and methods for their use are either known or readily ascertainable by those of skill in the art without undue experimentation. Similarly, methods and assays for screening a library of compounds for their ability to activate or inhibit P/Q channel activity and/or G_{αi} activity are widely known, and therefore, the identification of additional agonists/antagonists requires only routine experimentation by those of skill in the art in view of the instant teachings.

As described herein, an additional advantage of the instant methods is the biological application of fault diagnosis engineering for identification of critical molecules in complex signaling pathways. As such, the present methods provide for the identification of alternative, safe therapeutic routes and targets. For example, vulnerability analysis of the CREB pathway shows that CREB function is as vulnerable to the dysfunction of PKA as to the dysfunction of the P/Q channel and G_{αi} activity. Obviously it is difficult to target PKA for treatment of disorders related to the CREB function due to the fact that PKA function is essential for overall cellular function and viability. However, as described herein, there are alternative molecules such as the P/Q channel and G_{α1} that can be targeted, and therefore, be as effective as targeting PKA. In addition, the vulnerability assessment tool is also able to identify alternative targets for significantly modulating the activity of the output node that are safer and easier to target, and therefore, will reduce the likelihood of toxicity or undesirable side effects.

As noted herein, a variety of techniques and methods may be used to model and analyze biological systems as digital circuits according to the present disclosure. For example, the present disclosure is expressly not limited to fault analysis using the EPP method described herein. Furthermore, it is expressly noted that the systems and methods disclosed herein are not limited to the exemplary algorithms used in identifying and modeling feedback paths for a target system or those used in calculating vulnerability levels. The application of the systems and methods disclosed herein has the potential to improve biological understanding of the cellular-molecular system, from its very basic physiological condition to disease development, and ultimately to drug discovery.

As used herein, the term “P/Q calcium channel antagonist” and/or “Gai antagonist” is used generally to refer to an agent capable of direct or indirect inhibition of P/Q calcium channel, and/or Gai expression, translation, and/or activity; or P/Q calcium channel receptor and/or Gai receptor expression, translation, and/or activity. As used herein, the term “receptor” is used generally to refer to any molecular entity capable of binding or interacting (via covalent or non-covalent bonds) with P/Q calcium channel antagonist and/or Gai.

The term “P/Q calcium channel agonist” and/or “Gai agonist” and/or “P/Q calcium channel receptor agonist” and/or “Gai receptor agonist” is used generally to refer to an agent capable of direct or indirectly increasing P/Q calcium channel, and/or Gai expression, translation, and/or activity; or P/Q calcium channel receptor, and/or Gai receptor expression, translation, and/or activity. Many calcium channel and G_{αi} agonists and antagonists are currently available (e.g., omega-conotoxind, omega-agatoxin, verapamil, diltiazem, DHP, roscovitine, nifedipine, atropine, Bay K 8644; NEM, forskolin, cholera toxin, pertussis toxin, respectively), and methods for their use, including dosage amount and regime, are either known or readily ascertainable by those of skill in the art without undue experimentation. For example, in certain embodiments of the methods of the invention the following agents can be administered at the indicated dosage: Amlodipine: doses less than 40 mg/day for adult use and less than 0.3 mg/kg for pediatric use; Bepridil: doses less than 400 mg/day for adult use; Diltiazem: Adults doses of less than 360 mg/day (for immediate release formula) and less than 540 mg (for sustained release formula) and less than 360 mg/day (for IV injections) and less than 1 mg/kg for pediatric use; Felodipine: doses less than 10 mg/day for adult use and less than 0.3 mg/kg for pediatric use; flunarizine: doses less than 20 mg/day for adult use, Isradipine: doses less than 20 mg/day for adult use and less than 0.1 mg/kg for pediatric use; Nicardipine: Adults doses of less than 60 mg/day (for immediate release formula) and less than 120 mg (for sustained release formula) and less than 120 mg/day (for IV injections) and less than 1.25 mg/kg for pediatric use; Nifedipine: Adults doses of less than 90 mg/day (for immediate release formula) and less than 180 mg (for sustained release formula); Nimodipine: doses less than 360 mg/day for adult use; Nisoldipine: doses less than 60 mg/day for adult use; and Verapamil: Adults doses of less than 480 mg/day (for immediate release. formula) and less than 720 mg (for sustained release formula) and less than 0.15 mg/kg (for IV injections) and less than 2.5 mg/kg for pediatric use.

In an additional aspect, the invention relates to methods of treating and/or preventing neurological disorders, for example, learning and memory dysfunction, comprising administering to an individual an effective amount of a composition comprising a nucleic acid encoding or complementary to at least a portion of an P/Q calcium channel, and/or Gai; and/or a P/Q calcium channel receptor, and/or Gai receptor polypeptide, homolog, fragment or derivative thereof, in combination with a pharmaceutically acceptable carrier, wherein the composition is effective in treating and/or preventing said neurological pathology or condition. In certain embodiments, the neurological pathology or condition includes neuropathological disorders, for example, memory loss, Parkinson's, Alzheimer's, Huntington's disease, schizophrenia, dementia, and psychiatric or neurological conditions manifested by learning and memory dysfunction.

In any of the methods described herein, the nucleic acids or polypeptides of the invention may be delivered or administered in any pharmaceutically acceptable form, and in any pharmaceutically acceptable route as described in further detail below. For example, compositions comprising nucleic acids and/or polypeptides of the invention can be delivered systemically or administered directly to a cell or tissue for the treatment and/or prevention of a neurological pathology or condition. In certain additional embodiments, the nucleic acids and/or polypeptides of the invention comprise a carrier moiety that improves bioavailability, increases the drug half-life, targets the therapeutic to a particular cell or tissue type or combination thereof.

In additional aspects, the invention comprises methods of modulating the protein expression, activity, or transcription of calcium channels, and/or Gai; and/or an calcium channels receptor, and/or Gai receptor. In certain embodiments, the method comprises administering a recombinant nucleic acid encoding an calcium channels, and/or Gai; and/or an calcium channels receptor, and/or Gai receptor to a cell or tissue, in vitro, ex vivo, or in vivo. As discussed in detail below, the recombinant nucleic acid may be cistronic; i.e., comprise the desired coding sequence within a sing open reading frame (ORF); or it may contain one or more intronic sequences. In certain other embodiments, the method comprises administering a recombinant nucleic acid that is capable of hybridizing specifically to a nucleic acid that encodes an calcium channels, and/or Gai; and/or an calcium channels receptor, and/or Gai receptor polypeptide, to a cell or tissue, in vitro, ex vivo, or in vivo. The recombinant nucleic acid may also be incorporated into a vector nucleic acid, for example, a plasmid, viral vector, artificial chromosome or the like. In additional embodiments, a vector comprising a recombinant calcium channels, and/or Gai; and/or an calcium channels receptor, and/or Gai receptor encoding nucleic acid is contemplated. The nucleic acids of the invention may also contain one or more transcription or replication regulatory elements, selectable markers or translation modifying sequences operably linked to the coding nucleic acid.

In addition, the invention relates to nucleic acids, including interfering nucleic acids targeting calcium channels, and/or Gai; and/or a calcium channels receptor, and/or Gai receptor nucleic acids. For example, the present invention features a nucleic acid molecule, such as a decoy RNA, dsRNA, siRNA, shRNA, microRNA, aptamer, and/or antisense nucleic acid molecules, which down regulates expression of a sequence encoding an calcium channels, and/or Gai; and/or a calcium channels receptor, and/or Gai receptor proteins. In another embodiment, a nucleic acid molecule of the invention has an endonuclease activity or is a component of a nuclease complex, and cleaves RNA having a calcium channels, and/or Gai; and/or a calcium channels receptor, and/or Gai receptor nucleic acid sequence. In any of the interfering nucleic acid embodiments, the nucleic acid molecule comprises between 12 and 100 bases complementary to an RNA having a calcium channels, and/or Gai; and/or a calcium channels receptor, and/or Gai receptor nucleic acid sequence. In another embodiment, the nucleic acid molecule comprises between 14 and 24 bases complementary to an RNA having a calcium channels, and/or Gai; and/or a calcium channels receptor, and/or Gai receptor nucleic acid sequence. In any embodiment described herein, the nucleic acid molecule can be synthesized chemically according to methods well known in the art. A number of references describe useful methods and approaches for generating RNAs including: 6900187, 6383808, 7101991, 7285541, 7368436, 7022828; which are incorporated herein by reference.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted aGainst HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

Oligonucleotides (eg; antisense, GeneBlocs) are synthesized using protocols known in the art as described in Caruthers et al., 1992, Methods in Enzymology 211, 319, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

In one embodiment the invention relates to a method for treating or preventing a neurological pathology or condition by up-regulating the expression, transcription and/or activity of a gene encoding a calcium channels, and/or Gai; and/or a calcium channels receptor, and/or Gai receptor polypeptide. In one embodiment, inhibition or down-regulation with a nucleic acid molecule preferably is below that level observed in the presence of an inactive or attenuated molecule that is able to bind to the same site on the target RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of a calcium channels, and/or Gai; and/or a calcium channels receptor, and/or Gai receptor genes with the nucleic acid molecule of the instant invention is greater in the presence of the nucleic acid molecule than in its absence.

In certain aspects, the invention relates to diagnostic oligonucleotides and diagnostic oligonucleotide set(s), for which a correlation exists between the health status of an individual, and the individual's expression of RNA or protein products corresponding to the nucleotide sequence. In some instances, only one oligonucleotide is necessary for such detection. Members of a diagnostic oligonucleotide set may be identified by any means capable of detecting expression or a polymorphism of RNA or protein products, including but not limited to differential expression screening, PCR, RT-PCR, SAGE analysis, high-throughput sequencing, microarrays, liquid or other arrays, protein-based methods (e.g., western blotting, proteomics, mass-spectrometry, and other methods described herein), and data mining methods, as further described herein.

In the context of the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning-A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

The nucleic acid molecules of the instant invention, individually, or in combination or in conjunction with other drugs, can be used to treat diseases or conditions discussed above. For example, the subject can be treated, or other appropriate cells can be treated, as is evident to those skilled in the art, individually or in combination with one or more drugs under conditions suitable for the treatment.

In another aspect, the invention includes pharmaceutical compositions that include therapeutically- or prophylactically-effective amounts of a therapeutic and a pharmaceutically-acceptable carrier. The therapeutic can be a nucleic acid, e.g., a calcium channels, and/or Gai; and/or a calcium channels receptor, and/or Gai receptor nucleic acid, for example, a peptide nucleic acid, a cDNA, or RNA, such as for example, a small inhibitory RNA; a polypeptide comprising a portion of a calcium channels, and/or Gai; and/or a calcium channels receptor, and/or Gai receptor; or an antibody specific for a calcium channels, and/or Gai; and/or a calcium channels receptor, and/or Gai receptor polypeptide. In a further aspect, the invention includes, in one or more containers, a therapeutically- or prophylactically-effective amount of this pharmaceutical composition. Also included in the invention is an oligonucleotide, e.g., an oligonucleotide which includes at least 6 contiguous nucleotides of a calcium channels, and/or Gai; and/or a calcium channels receptor, and/or Gai receptor nucleic acid or a complement of said oligonucleotide.

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

The molecules of the instant invention can be used as pharmaceutical agents. Pharmaceutical agents prevent, inhibit the occurrence, or treat (alleviate a symptom to some extent, preferably all of the symptoms) a disease state in a subject. A number of useful nucleic acid-based therapeutic approaches are known and discussed in Patil et al., AAPS Journal, 2005; 7(1):E61-77, which is incorporated by reference in its entirety.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols. Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle. The amount of active ingredient that can be combined with the carrier materials to produce a single dosage form varies depending upon the host treated and the particular mode of administration. Dosage unit forms generally contain between from about 1 mg to about 5000 mg of an active ingredient. It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; propulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al, 1992, Nucleic Acids Res., 20, 4581 9; Yu et al., 1993, Proc. Natl. Acad. Sci. USA, 90, 6340 4; L′Huillier et al., 1992, EMBO J., 11, 4411 8; Lisziewicz et al., 1993, Proc. Natl. Acad. Sci. U.S.A, 90, 8000 4; Thompson et al., 1995, Nucleic Acids Res., 23, 2259; Sullenger & Cech, 1993, Science, 262, 1566).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment, an isolated nucleic acid molecule of the invention comprises a nucleic acid molecule that is a complement of the nucleotide sequence of a calcium channels, and/or Gai; and/or a calcium channels receptor, and/or Gai receptor nucleic acid. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In a further aspect, the invention includes methods of producing a polypeptide by expressing, in a cell, an endogenous or exogenous calcium channels, and/or Gai; and/or a calcium channels receptor, and/or Gai receptor nucleic acid. If desired, the polypeptide can then be recovered. In still another aspect, the invention includes a method of producing a polypeptide by culturing a cell that contains an endogenous nucleic acid encoding a calcium channels, and/or Gai; and/or a calcium channels receptor, and/or Gai receptor nucleic acid, disposed upstream or downstream of an exogenous regulatory element, for example, a promoter, enhancer or repressor sequence. In certain embodiments, the exogenous regulatory element is incorporated into a host cell's genome through homologous recombination, strand break or mismatch repair mechanisms which are widely known in the art.

In a further aspect, the invention provides a method for modulating the activity or expression of aa calcium channels, and/or Gai; and/or a calcium channels receptor, and/or Gai receptor polypeptide, by contacting a cell sample that includes the calcium channels, and/or Gai; and/or a calcium channels receptor, and/or Gai receptor polypeptide with a compound that binds to the calcium channels, and/or Gai; and/or a calcium channels receptor, and/or Gai receptor polypeptide, a calcium channels, and/or Gai; and/or a calcium channels receptor, and/or Gai receptor RNA binding protein, and/or a calcium channels, and/or Gai; and/or a calcium channels receptor, and/or Gai receptor protein interactor in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Any of the embodiments described herein may contain an additional therapeutic agent. In additional embodiments, therapeutics of the invention may comprise one or more biologically active ingredients such as, Analgesics, Antacids, Antianxiety Drugs, Antiarrhythmics, Antibacterials, Antibiotics, Anticoagulants and Thrombolytics, Anticonvulsants, Antidepressants, Antidiarrheals, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Cold Cures, Corticosteroids, Cough Suppressants, Cytotoxics, Decongestants, Diuretics, Expectorants, Hormones, Hypoglycemics (Oral), Immunosuppressives, Laxatives, Muscle Relaxants, Sedatives, Sex Hormones, Sleeping Drugs, Tranquilizer, Vitamins or a combination thereof.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

In yet another aspect, the invention includes a method for determining the presence of or predisposition to a disease associated with a muscle-related pathology or muscle dysfunction in a subject (e.g., a human subject). The method comprises detecting the genotype or haplotype of a calcium channels, and/or Gai; and/or a calcium channels receptor, and/or Gai receptor gene by treating a tissue sample from an individual with a detectable probe specific for a calcium channels, and/or Gai; and/or a calcium channels receptor, and/or Gai receptor polymorphism or mutation, and detecting the formation of a probe/target complex, wherein formation of a complex is indicative of the presence of a particular genotype. Alternatively, measuring the amount of a calcium channels, and/or Gai; and/or a calcium channels receptor, and/or Gai receptor nucleic acid or polypeptide in a test sample from the subject and comparing the amount of the polypeptide in the test sample to the amount of a calcium channels, and/or Gai; and/or a calcium channels receptor, and/or Gai receptor nucleic acid or polypeptide present in a control sample. An alteration in the level in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various disorders as well as to determine the stage of particular disorders.

In another aspect, the invention relates to a method for diagnosing or monitoring disorder or disease or progression comprising detecting for the presence of a nucleotide polymorphism in a calcium channels, and/or Gai; and/or a calcium channels receptor, and/or Gai receptor gene, associated with a disease, through the detection of the presence of a calcium channels, and/or Gai; and/or a calcium channels receptor, and/or Gai receptor nucleic aid, protein or both; the transcription of a calcium channels, and/or Gai; and/or a calcium channels receptor, and/or Gai receptor nucleic aid, protein or both; or expression level of a calcium channels, and/or Gai; and/or a calcium channels receptor, and/or Gai receptor nucleic aid, protein or both.

In an embodiment, the invention comprises a method for screening for agents that modulate at least one of a calcium channels, and/or Gai; and/or a calcium channels receptor, and/or Gai receptor activity, protein levels, or gene expression comprising providing a cell or tissue; measuring for the amount of at least one of endogenous a calcium channels, and/or Gai; and/or a calcium channels receptor, and/or Gai receptor activity, protein level, or gene expression to establish a control value; contacting a test agent to the cell or tissue; measuring or detecting the activity of at least one of a calcium channels, and/or Gai; and/or a calcium channels receptor, and/or Gai receptor, amount of a calcium channels, and/or Gai; and/or a calcium channels receptor, and/or Gai receptor protein, or amount of a calcium channels, and/or Gai; and/or a calcium channels receptor, and/or Gai receptor gene expression to establish a test value; and comparing the control value to the test value, wherein an observed change between the test and control values indicates an agent capable of modulating at least one of a calcium channels, and/or Gai; and/or a calcium channels receptor, and/or Gai receptor activity, protein levels, or gene expression in the cell or tissue.

The invention further includes a method for screening for a modulator of disorders or syndromes including, e.g., the diseases and disorders disclosed above and/or other pathologies and disorders of the like. The method includes contacting a test compound with a calcium channels, and/or Gai; and/or a calcium channels receptor, and/or Gai receptor nucleic acid or polypeptide, and determining if the test compound binds to said a calcium channels, and/or Gai; and/or a calcium channels receptor, and/or Gai receptor nucleic acid or polypeptide. Binding of the test compound to the a calcium channels, and/or Gai; and/or a calcium channels receptor, and/or Gai receptor nucleic acid or polypeptide indicates the test compound is a modulator of activity, transcription, translation or of latency or predisposition to the aforementioned disorders or syndromes.

In still additional aspects, the invention relates to methods of screening for compounds that modulate neurological and/or learning and memory function by contacting at least one of a calcium channels, and/or Gai; and/or a calcium channels receptor, and/or Gai receptor nucleic acid or polypeptide a combination thereof, with a test compound; and measuring the binding of the test compound, and/or the effects on neurological and/or learning and memory function.

Libraries of potential compounds are widely known and readily available that could be used in the methods of the invention. Furthermore, the techniques useful for measuring the binding of agents to a calcium channels, and/or Gai; and/or a calcium channels receptor, and/or Gai receptor polypeptides, the amount of a calcium channels, and/or Gai; and/or a calcium channels receptor, and/or Gai receptor protein, and/or the level of a calcium channels, and/or Gai; and/or a calcium channels receptor, and/or Gai receptor gene transcription and/or translation are described herein. Additional methods useful for practicing the invention are routinely used and can be adapted for use in the claimed methods using routine experimentation for the art.

In certain additional aspects the invention relates to compositions and methods related to the treatment of neurological pathologies and conditions. In certain exemplary embodiments, the invention encompasses, for example, the administration of an effective amount of a therapeutic composition of the invention to an individual for the treatment and/or prevention of neurological and/or learning and memory-related pathologies and conditions; treatment and/or prevention of aneurological and/or learning and memory-related; treatment and/or prevention of injury to any type of neurological and/or learning and memory-related, such as those occurring in subjects suffering from sports-related injuries; age-related neurological and/or learning and memory-related dysfunction, ischemia, stroke, viral/bacterial/parasitic infectionsor, Alzheimer's disease, stroke, Parkinson's disease, Huntington's disease, cerebral palsy, epilepsy, Lesch-Nyhan syndrome, multiple sclerosis, ataxia-telangiectasia, behavioral disorders, addiction, anxiety, neurodegenerative disorders, neurologic disorders, developmental defects, conditions associated with the role of GRK2 in brain and in the regulation of chemokine receptors, encephalomyelitis, anxiety, schizophrenia, manic depression, delirium, dementia, severe mental retardation and dyskinesias, and/or other pathologies and disorders of the like.

In certain aspects, the modulation of neurological function is accomplished by, for example, the modulation of a calcium channels, and/or Gai; and/or a calcium channels receptor, and/or Gai receptor nucleic acids or polypeptides, and/or a calcium channels, and/or Gai; and/or a calcium channels receptor, and/or Gai receptor nucleic acid or polypeptide binding partners, i.e., modulation of factors that bind to calcium channels, and/or Gai; and/or a calcium channels receptor, and/or Gai receptor nucleic acids and/or a calcium channels, and/or Gai; and/or a calcium channels receptor, and/or Gai receptor polypeptides, and inhibit, attenuate or neutralize their biological activities, such as at least one a calcium channels, and/or Gai; and/or a calcium channels receptor, and/or Gai receptor binding protein, inhibitory RNA, antibody, pseudopeptide, peptide analog or peptidomimetic, or small molecule that bind and inhibit one or more target nucleic aids or polypeptide.

In another aspect the present invention provides a kit comprising a suitable container, a composition of the invention disposed therein, and instructions for its use. A further object of the present invention is to provide a kit comprising a suitable container, a therapeutic of the invention in a pharmaceutically acceptable form disposed therein, and instructions for its use. Also disclosed according to the present invention is a kit or system utilizing any one of the methods, selection strategies, materials, or components described herein. Exemplary kits according to the present disclosure will optionally, additionally include instructions for performing methods or assays, packaging materials, one or more containers which contain an assay, a device or system components, or the like.

The therapeutic compositions of the invention comprise, in certain embodiments, for example, a nucleic acid encoding a calcium channels, and/or Gai; and/or a calcium channels receptor, and/or Gai receptor polypeptide, a calcium channels, and/or Gai; and/or a calcium channels receptor, and/or Gai receptor nucleic acid; a nucleic acid that binds a nucleic acid encoding a calcium channels, and/or Gai; and/or a calcium channels receptor, and/or Gai receptor polypeptide; a calcium channels, and/or Gai; and/or a calcium channels receptor, and/or Gai receptor encoding nucleic acid; a calcium channels, and/or Gai; and/or a calcium channels receptor, and/or Gai receptor peptide analog, pseudopeptide or peptidomimetic based thereon; a small molecule modulator of a calcium channels, and/or Gai; and/or a calcium channels receptor, and/or Gai receptor or a calcium channels, and/or Gai; and/or a calcium channels receptor, and/or Gai receptor protein-protein interaction; or a calcium channels, and/or Gai; and/or a calcium channels receptor, and/or Gai receptor-specific antibody or biologically-active derivatives or fragments thereof. As described herein, a calcium channels, and/or Gai; and/or a calcium channels receptor, and/or Gai receptor play an important role in normal neurological function. Therefore, targeting the expression and/or activity of these nucleic acids, polypeptides, and homologs thereof will allow for a novel treatment of various acute and chronic diseases and conditions related to neurological dysfunction and degeneration.

In any aspect of the invention, the therapeutic composition of the invention can be in any pharmaceutically acceptable form and administered by any pharmaceutically acceptable route, for example, the therapeutic composition can be administered as an oral dosage, either single daily dose or unitary dosage form, for the treatment of a muscle damage due to a myocardial infarction, sclerotic lesion, or muscle tear due to sports-related activity to promote the regeneration and repair of the damaged muscle tissue. Such pharmaceutically acceptable carriers and excipients and methods of administration will be readily apparent to those of skill in the art, and include compositions and methods as described in the USP-NF 2008 (United States Pharmacopeia/National Formulary), which is incorporated herein by reference in its entirety.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The active compounds will generally be formulated for parenteral administration, e.g., formulated for injection via the intravenous, intraarthricular, intrathecal, intramuscular, sub-cutaneous, intra-lesional, or even intraperitoneal routes. The preparation of an aqueous composition that contains a cancer marker antibody, conjugate, inhibitor or other agent as an active component or ingredient will be known to those of skill in the art in light of the present disclosure. Typically, such compositions can be prepared as injectibles, either as liquid solutions or suspensions; solid forms suitable for using to prepare solutions or suspensions upon the addition of a liquid prior to injection can also be prepared; and the preparations can also be emulsified.

A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The compounds, nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

An effective amount, pharmaceutically effective dose, therapeutically effective amount, or pharmaceutically effective amount is that dose required to prevent, inhibit the occurrence, or treat (alleviate a symptom to some extent, preferably all of the symptoms) of a disease state or pathological condition. The effective amount depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer. In addition, effective amounts of the compositions of the invention encompass those amounts utilized in the examples to facilitate the intended or desired biological effect.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The formulations can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release of the active compound. For buccal administration the compositions may take the form of tablets or lozenges formulated in conventional manner.

Excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present. Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for injectable use include sterile aqueous solutions (where water soluble) or dispersions and sterile powders for the extemporaneous preparation of sterile injectable solutions or dispersion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved aGainst the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In one embodiment, the active compounds are prepared with carriers that will protect the compound aGainst rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For administration to non-human animals, the therapeutic compositions of the invention can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water. The composition can also be administered to a subject in combination with other therapeutic compounds to increase the overall therapeutic effect. The use of multiple compounds to treat an indication can increase the beneficial effects while reducing the presence of side effects.

Although the present disclosure has been described with reference to exemplary embodiments and implementations thereof, the present disclosure is not to be limited by or to such disclosed embodiments and/or implementations. Rather, the disclosed fault diagnosis methods have wide ranging applications, and are susceptible to many variations, modifications and/or enhancements without departing from the spirit or scope hereof. The present disclosure expressly encompasses all such variations, modifications and/or enhancements.

### EXAMPLES

We constructed a complex neuronal network, following the approach of Ma'ayan et al. (2). The output node is the transcription factor CREB (cAMP responsive element binding protein) and the input nodes are seven major ligands in nervous system—glutamate, dopamine, GABA, serotonin, ACh, adenosine, and enkephalin. The constructed CREB network (1) comprised of 64 molecules and 152 intermolecular interactions (1). The logic equations for the CREB network (1) were derived using Rule #1 and Rule #2 (1), and the corresponding digital electronic circuit, was constructed (1). Calculation of the vulnerabilities of all the molecules revealed that dysfunctional in 41 molecules out of 64 would not contribute to the failure of CREB circuit (vulnerability values less than 0.1).

The molecules that showed a vulnerability of more than 0.5, which indicates that their dysfunction can result in the failure of CREB function, were calmodulin, calcium, cAMP, Gαᵢ, adenlyate cyclase 1 (AC1), AC2, AC5, protein kinase A (PKA), L AND/OR P/Q-type calcium channel (L AND/OR P/Q CaCh), and PP2A (1). These molecules can be grouped into elements of the cAMP-dependent signaling (cAMP, Gαᵢ, PKA, and the AC isoforms) and elements of calcium signaling (calcium, calmodulin, and L AND/OR P/Q-type calcium channels). The molecules with the highest vulnerability values in the CREB network are functionally related molecules, and some of them were already known as main physiological regulators of CREB function. Indeed the name CREB is based on the identification of the protein as a cAMP responsive element binding protein. This engineering analysis identified cAMP and the molecules directly related to cAMP function, such as AC1, AC2, AC5, and PKA, as the most critical molecules for the regulation of CREB (1). The crucial role of PKA in the regulation of CREB is well known (3). Important functions of the cognitive and executive human brain, such as learning and memory, are directly regulated by cAMP-dependent CREB functions (3). In pathological terms, direct evidence for deregulation of PKA signaling has been reported in human disorders manifested by learning and memory dysfunction, such as Alzheimer Disease (7) or schizophrenia (8, 16). Vulnerability assessment of the CREB circuit also identified some elements of calcium signaling as playing a major role in the function of CREB. This observation was also physiologically and pathologically relevant and consistent with experimental data (9). Furthermore, several pathological conditions associated with learning and memory dysfunction can arise from deregulation of calcium-dependent signaling (10-12).

Because many of the identified molecules were already experimentally known regulator of CREB, here we provide experimental evidence for only two, Gαᵢ and L AND/OR P/Q-type calcium channels. We altered the activity or abundance of endogenous L AND/OR P/Q-type calcium channels and Gαᵢ in primary cortical neurons from rats and then assayed for the changes in endogenous CREB activity or abundance (see Methods). We used primary neuronal culture as a model system because it has the most similar CREB signaling network to the in vivo signaling networks of the mammalian brain.

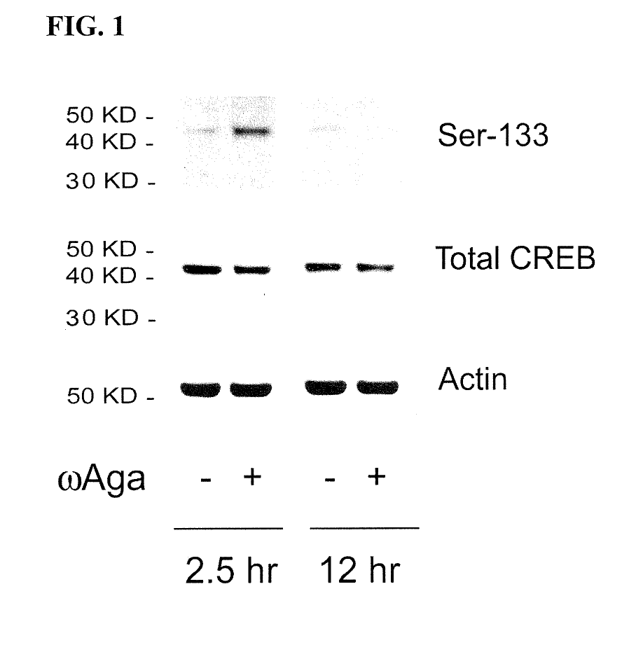

Short-term (2.5 h) treatment of primary neurons with the selective L AND/OR P/Q-type calcium channel blocker, ω-Agatoxin IVA, increased the phosphorylation of CREB at Ser¹³³, without changing the total abundance of CREB (FIG. 1A). In contrast, long-term (12 h) treatment decreased the phosphorylation of CREB at Ser¹³³, as well as decreased the abundance of CREB (FIG. 1A). Furthermore, we targeted the expression of L AND/OR P/Q-type calcium channels with three unique adenoviral shRNA constructs and measured their effect on the endogenous total protein and the phosphorylation levels of CREB at Ser¹³³. Three unique adenoviral shRNA expression vectors (CACNA1A V1, V2, and V3), which target the expression of transcript variants 1 and 2 of the alpha 1A subunit of L AND/OR P/Q-type calcium channels were used to knockdown the expression of the channels in neurons. The V2 vector caused a substantial decrease in the abundance of L AND/OR P/Q-type calcium channels, the V1 vector caused a smaller decrease in the abundance, and V3 did not affect the abundance of the channel in primary neurons. We observed a decrease in the abundance of CREB and in the proportion of Ser¹³³ phosphorylated CREB in cells with the V2 vector. However, the V3 vector, which did not alter L AND/OR P/Q-type calcium channel abundance, did not affect the abundance or phosphorylation of CREB. These experiments show that the activity or abundance of L AND/OR P/Q-type calcium channels can affect both the abundance and phosphorylation state of CREB. This observation is consistent with the findings of Sutton et al. (4), which shows exogenous expression of L AND/OR P/Q-type calcium channels can induce transcription of specific genes related to the synaptic function. Whether L AND/OR P/Q-type calcium channels are altering calcium signaling to affect CREB activity or whether they are functioning through a nonconducting mechanism, similar to that described for other types calcium channels (L and N type) (5), remains to be determined. It is also an open question as to why there are opposing effects of short-term and long-term blockage of L AND/OR P/Q-type calcium channel activity on CREB phosphorylation. A possible biological explanation for the different response between the short and long term treatments is related to the compensatory mechanisms that re-regulate the initial cellular response. The existing feedbacks in the circuit may explain the role of compensatory mechanisms that occur within different time intervals in biological systems. More detailed studies are needed to precisely address the differences among short and long term responses.

To verify the importance of Gαᵢ in the regulation of CREB we inhibited Gαᵢ with pertussis toxin (6) (see Methods) and found an increase in the abundance CREB and an increase in Ser¹³³ phosphorylation. In contrast, activation of Gαᵢ by MAS-7, an active mastoparan analog that stimulates Gαᵢ (6), caused a decrease in the abundance of CREB and a decrease in Ser¹³³ phosphorylation (FIG. 1C). The decrease in abundance of CREB, along with the decrease in Ser¹³³ phosphorylation, is consistent with an overall decrease in the activity of CREB, as phosphorylation of CREB at Ser¹³³ is required for activity (3). These experiments demonstrate the ability of the fault diagnosis engineering approach to correctly identify key regulators of signaling pathways.

With the primary neuronal cultures, we tested the ability of the constructed Boolean network to correctly predict the output activity, following an increase in the concentration of each input molecules. According to the logic equations of the CREB network (1), which provide the on/off state of CREB in response to the on/off states of input neurotransmitters (1), serotonin is the only input activates CREB. To verify this prediction experimentally, we treated primary neurons with equal micromolar concentrations (10 μM) of serotonin, glutamate, dopamine, GABA, and adenosine for 15 minutes and measured the activity of CREB by monitoring Ser133 phosphorylation (14). Receptors of these neurotransmitters are expressed in the primary cortical culture and this concentration is used in the literature to activate these receptors (17-22). As shown in FIG. 1D, treatment with serotonin had the most robust effect on the activity of CREB, stimulating phosphorylation to a similar extent as did the PKA activator forskolin. However, exposure of the neurons to the other ligand failed to robustly activate CREB (FIG. 1D). This is consistent with the output predicted by the logic equations: Serotonin should beon the most effective activator of CREB. Previous studies have also reported that serotonin can induce Ser133 CREB phosphorylation (18), thus the engineering model not only correctly predicts this effect of serotonin, but also shows the specificity of serotonin's effect, compared to the other ligands.

We also tried to verify the biological relevance of the stuck-at-1 and stuck-at-0 fault models used in Abdi et al (1). We tested the effect of serotonin on the activity of CREB when PKA, a highly vulnerable molecule in this network, is either activated (stuck-at-1) or inhibited (stuck-at-0), by forskolin or H-89, respectively. When PKA is stuck-at-1 or 0, the model predicts that serotonin should no longer activate CREB (1). To verify this experimentally, we treated primary neurons with serotonin in the presence or absence of either forskolin or H-89 (FIG. 1E). Whereas individual treatments with serotonin or forskolin activate CREB, treatment with serotonin following activation of PKA by forskolin did not affect the activity of CREB, as compared to treatment with serotonin only. Thus, when the highly vulnerable molecule PKA is stuck-at-1, the network output is not correctly regulated by the input. Inhibition of PKA by H-89 also prevents serotonin from stimulating the activity of CREB (FIG. 1E). Immunofluorescent analysis of CREB phosphorylation in primary neuron cultures exposed to vehicle, forskolin, or serotonin individually, or to serotonin following treatment with forskolin, was consistent with the results obtained by Western blotting for CREB phosphorylation. The cells were triple labeled with antibodies against Ser133 CREB (as a measure of CREB activity), Map-2 (as a marker that specifically labels neurons), and DRAQ-5 (as a nuclear marker that stains the nucleus of neuronal and normeuronal cells in primary culture). Treatment of the neuronal primary cultures with forskolin increased the activity of CREB in both neuronal and non-neuronal (glial) cells in primary culture. However, treatment with serotonin only increased the activity of CREB in the neurons, not in the normeuronal cells. This specific activation in the neuronal cell population by serotonin was expected, because neurons have receptors for serotonin whereas the normeuronal cells in the culture do not. Consistent with the Western blot data (FIG. 1E), the effect of serotonin on the activity of CREB in neurons is attenuated, when serotonin was added after treatment with forskolin. These two experiments suggest that dysfunction of the network output occurs when PKA is stuck in either an active or inactive state. Thus, the data support the prediction of the model that serotonin fails to activate CREB when PKA is faulty, which is entirely consistent with PKA having a high vulnerability value in the network.

Methods

Primary cortical culture. Primary cortical cultures were prepared from brains of embryonic day 17 to 18 Sprague-Dawley rats (Charles River). After trituration of cortical sections with a glass pipette, 2 to 4×10⁵ neurons were plated on a coverslip (diameter: 12 mm) precoated with poly d-lysine (BD Biocoat). For biochemical analysis, primary cortical cells were plated in 35 mm dishes precoated with poly d-lysine (˜3×10⁶ neurons/dish). Neurons were grown in NEUROBASAL Medium supplemented with 0.5 mM L-glutamine, B27 (2%), and N2 (1%) supplements.

Protein extraction, and immunoblot analysis. Primary neurons were cultured as described. Cells were homogenized in ice cold lysate buffer (0.25 M Tris, pH 7.5) containing protease inhibitors (Protease Inhibitor Cocktail tablets, Boehringer Mannheim) and phosphatase inhibitors (Phosphatase Inhibitor Cocktails I & II, Sigma) and lysed through three cycles of freezing (in liquid nitrogen) and thawing (in 37° C. water bath). Protein concentration was measured by Bio-Rad's protein assay and spectrometry at 595° A. Equal amounts of total protein were loaded on 4-12% gradient Bis-Tris gels, separated using the NuPAGE system (Invitrogen) and transferred onto nitrocellulose membrane. The membrane was probed with primary and secondary antibodies and signals were detected by chemiluminescence followed by autoradiography. The following antibodies were used: anti-CREB antibody (Cell Signaling, 1:1000), anti-phospho Ser¹³³ CREB antibody (Cell Signaling, 1:1000), anti P/Q type calcium channel antibody (Chemicon, 1:1000), and anti-actin antibody (Sigma, 1:1000).

Immunofluorescent studies. Cortical neurons were grown on coverslips, fixed for 10 min in PBS plus 3.7% formaldehyde, and permeabilized for 2 min with cold acetone. Coverslips were coated with 1000 of primary antibody diluted in PBS (anti Phospho Ser¹³³ CREB, Cell Signaling, 1:200, anti Map-2 antibody, Upstate Biotechnology, 1:250). Coverslips washed three times and labeled with Alexa 568 anti-rabbit antibody (1:500), Alexa 488 anti-mouse:antibody (1:500), and the nuclear marker DRAQ-5 (1:10,000).

Analysis of CREB regulation by P/Q-type calcium channels or Gαᵢ. Primary cortical neurons were cultured as described. We followed the methods of Dolmetsch et al. (13) to analyze the effect of calcium channel blockers on CREB activity. We characterized the signaling properties of the endogenous P/Q-type calcium channels by monitoring the endogenous activity and total protein levels of CREB. In order to minimize the effect of other elements of calcium or G protein signaling following neuronal depolarization, all experiments were performed without depolarizing the neurons. Previous time-course studies have shown that short-term Ser¹³³ phosphorylation of CREB are transient events and prolonged Ser¹³³ phoaphorylation (more than 40 minutes) is required for transcriptional activity (13, 14, 15). Therefore, in these experiments we analyzed the endogenous Ser¹³³ phosphorylation and total CREB protein abundance following at least 2 hours treatment with agonist or antagonists of P/Q-type calcium channels and 2 hours treatment with agonist or antagonist of Gαᵢ, ω-Agatoxin IVA (Calbiochem), pertussis toxin (Sigma), and Mas-7 (Calbiochem) were dissolved in the appropriate vehicle and added to the medium. Following the treatment, cortical neurons were harvested, lysed, and subjected to Western blot analysis as described.

Adenoviral gene knockdown. We used AdenoSilence™ RNAi viral vector kit (Millipore, Cat# GAL10021) to target the expression of P/Q-type calcium channels in primary neuronal cultures. This kit consisted of three unique adenoviral shRNA constructs targeting the transcript variants 1 and 2 of the alpha 1A subunit of P/Q-type calcium channels (CACNA1A). Following the kit instruction, 60×10⁶ VPU (Viral Particle Unit) from the crude virus of each construct was added to 50 ul of complete medium and added to 35-mm dishes of primary neuronal cultures. Cells were harvested 48 hours after viral transduction, protein was extracted, and the lysates were subject to Western blot analysis.

### REFERENCES

The following references are incorporated herein by reference, in their entirety for all purposes.
1. Abdi, A., Tahoori, M. B., Emamian, E. S. Fault diagnosis engineering of digital circuits can identify vulnerable molecules in complex cellular pathways. Sci. Signal 1, ra8 (2008).
2. Ma'ayan, A. et al. Formation of regulatory patterns during signal propagation in a mammalian cellular network. Science 309: 1078-1083 (2005).
3. Lonze, B. E. & Ginty, D. D. Function and regulation of CREB family transcription factors in the nervous system. Neuron 35: 605-623 (2002).
4. Sutton, K. G., McRory, J. E., Guthrie, H., Murphy, T. H. & Snutch, T. P. P/Q-type calcium channels mediate the activity-dependent feedback of syntaxin-1A. Nature 401: 800-804 (1999).
5. Ikeda, S. R. Signal transduction: calcium channels—Link locally, act globally. Science 294: 318-319 (2001).
6. Jena, B. P. et al. Gi regulation of secretory vesicle swelling examined by atomic force microscopy. Proc. Natl. Acad. Sci. USA 94: 13317-13322 (1997).
7. Jicha, G. A. et al. cAMP-dependent protein kinase phosphorylations on tau in Alzheimer's disease. J. Neurosci. 19: 7486-7494 (1999).
8. Millar, J. K. et al. DISC1 and PDE4B are interacting genetic factors in schizophrenia that regulate cAMP signaling. Science 310: 1187-1191 (2005).
9. West, A. E. et al. Calcium regulation of neuronal gene expression. Proc. Nail. Acad. Sci. USA 98: 11024-1031 (2001).
10. Lidow, M. S. Calcium signaling dysfunction in schizophrenia: a unifying approach. Brain Res Rev. 43: 70-84 (2003).
11. Tu, H. et al. Presenilins form ER Ca2+ leak channels, a function disrupted by familial Alzheimer's disease-linked mutations. Cell 126: 981-993 (2006).
12. Hans, M. et al. Functional consequences of mutations in the human alpha1A calcium channel subunit linked to familial hemiplegic migraine. J. Neurosci. 19: 1610-1619 (1999).
13. Dolmetsch, R. E. et al. Signaling to the nucleus by an L-type calcium channel-calmodulin complex through the MAP kinase pathway. Science. 294, 333 (2001).
14. D. D. Ginty et al., Regulation of CREB phosphorylation in the suprachiasmatic nucleus by light and a circadian clock. Science 260, 238 (1993).
15. H. Bito, K. Deisseroth, R. W. Tsien, CREB phosphorylation and dephosphorylation: a Ca(2+)- and stimulus duration-dependent switch for hippocampal gene expression. Cell 87, 1203 (1996).
16. Piskulic, D., Olver, J. S., Norman T. R., & Maruff, P. Behavioural studies of spatial working memory dysfunction in schizophrenia: a quantitative literature review. Psychiatry Res. 150: 111-121 (2007).
17. Goldman-Rakic, P. S., Lidow, M. S., & Gallager, D. W. Overlap of dopaminergic, adrenergic, and serotoninergic receptors and complementarity of their subtypes in primate prefrontal cortex. J. Neurosci. 10: 2125-2138 (1990).
18. Mahgoub, M. A., Sara, Y., Kavalali, E. T., & Monteggia, L. M. Reciprocal interaction of serotonin and neuronal activity in regulation of cAMP-responsive element-dependent gene expression. J Pharmacol Exp Ther. 317: 88-96 (2006).
19. Alagarsamy, S., Phillips, M., Pappas, T., & Johnson, K. M. Dopamine neurotoxicity in cortical neurons. Drug Alcohol Depend. 48: 105-111 (1997).
20. Fatatis, A., & Russell, J. T. Spontaneous changes in intracellular calcium concentration in type 1 astrocytes from rat cerebral cortex in primary culture. Glia. 5: 95-104 (1992).
21. Yu, R., Xu, X. H., & Sheng, M. P. Differential effects of allopregnanolone and GABA on kainate-induced lactate dehydrogenase release in cultured rat cerebral cortical cells. Acta Pharmacol Sin. 23: 680-684 (2002).
22. Nicolas, F., Oillet, J., Koziel, V., & Daval, J. L. Characterization of adenosine receptors in a model of cultured neurons from rat forebrain. Neurochem Res. 19: 507-515 (1994).

What is claimed is:

1. A method of modulating cAMP response element binding protein (CREB) activity comprising the steps of administering a composition comprising an effective amount of an agent that modulates the activity, expression, or levels of at least one of:
a calcium channel;
an inhibitory Guanine nucleotide-binding protein (G protein);
protein phosphatase 2A (PP2A); or
calcium/calmodulin dependent kinase (CaM kinase), wherein the agent is effective in modulating CREB activity.

2. The method of claim 1, wherein the neurological function to be modulated is a learning and memory related disorder or learning and memory related dysfunction.

3. The method of claim 1, wherein the agent is an agonist or antagonist of an L-type calcium channel or a P/Q type calcium channel or a combination thereof.

4. The method of claim 1, wherein the agent is an agonist or antagonist of Gai G protein.

5. The method of claim 1, wherein the agent is an agonist or antagonist of PP2A.

6. The method of claim 1, wherein the agent is an agonist of CaM kinase.

7. The method of claim 1, wherein the agent is at least one agent selected from the group consisting of Amlodipine, Bepridil, Diltiazem, Felodipine, Flunarizine, Isradipine, Nicardipine, Nifedipine, Nimodipine, Nisoldipine, and Verapamil.

8. The method of claim 7, wherein the effective amount is from 0.0001 mg/kg and 100 mg/kg body weight/day.

9. The method of claim 1, wherein the composition further comprises a pharmaceutically acceptable carrier or excipient.

10. The method of claim 1, wherein the agent is a nucleic acid capable of specifically hybridizing to an mRNA transcript encoding a protein selected from the group consisting of a calcium channels; Gαi; PP2A; and CaM kinase.

11. The method of claim 10, wherein the nucleic acid is at least one of an inhibitory RNA or an antisense RNA or a composition comprising both.

12. The method of claim 11, wherein the inhibitory RNA is a microRNA.

13. A vector comprising the nucleic acid of claim 11, operably linked to a transcription regulatory nucleic acid sequence.

14. A method for the treatment of learning and memory dysfunctions comprising the steps of administering a composition comprising an effective amount of an agent that modulates the activity, expression, or levels of at least one of:
a calcium channel;
an inhibitory Guanine nucleotide-binding protein (G protein);
a protein phosphatase 2A (PP2A); or
a calcium/calmodulin dependent kinase (CaM kinase), wherein the agent is effective in modulating CREB activity.

15. The method of claim 14, wherein the agent is an agonist or antagonist of an L-type calcium channel or a calcium channels or a combination thereof.

16. The method of claim 14, wherein the agent is an agonist or antagonist of Gai G protein.

17. The method of claim 14, wherein the agent is an agonist or antagonist of PP2A.

18. The method of claim 14, wherein the agent is an agonist of CaM kinase.

19. The method of claim 14, wherein the agent is at least one agent selected from the group consisting of Amlodipine, Bepridil, Diltiazem, Felodipine, Flunarizine, Isradipine, Nicardipine, Nifedipine, Nimodipine, Nisoldipine, and Verapamil.

20. A method for screening for agents that modulate CREB comprising the steps of providing a cell or tissue and measuring for the activity, expression, or protein level of CREB to obtain a control value; testing for agents that modulate at least one of the activity, expression or protein level of at least one member selected from the group consisting of a calcium channels, Gai, PP2A, and CaM kinase; contacting the cell or tissue having CREB activity, expression or protein levels with an agent capable or modulating at least one of the activity, expression or protein level of at least one member selected from the group consisting of a calcium channels; Gαi; PP2A; and CaM kinase to obtain a test value; and comparing the control value to the test value, wherein an observed change between the test and control values indicates an agent capable of modulating at least one of the activity, expression, or protein level of CREB.
